SEQUENCE LISTING (6 sequences)

SEQ ID NO: 1 - DNA, 27 bp, Hepatitis B virus
cctcagtccgtttctcctggctcagtt

SEQ ID NO: 2 - DNA, 19 bp, Hepatitis B virus
ccccttcttcgtctgccgt

SEQ ID NO: 3 - DNA, 20 bp, Hepatitis B virus
  primer_bind (1)..(20)
tgcacctgtattcccatccc

SEQ ID NO: 4 - DNA, 26 bp, Hepatitis B virus
  primer_bind (1)..(26)
ccacatcatccatataactgaaagcc

SEQ ID NO: 5 - DNA, 16 bp, Hepatitis B virus
  primer_bind (1)..(16)
cgtcggcgctgaatcc

SEQ ID NO: 6 - DNA, 18 bp, Hepatitis B virus
  primer_bind (1)..(18)
gaagcgaagtgcacacgg

(19) United States
(12) Patent Application Publication
(10) Pub. No.: US 2011/0306037 A1
(43) Pub. Date: Dec. 15, 2011

(54) OLIGONUCLEOTUIDE PROBES AND PRIMERS FOR DETECTION OF HEPATITIS B VIRUS

(75) Inventors: Manjula Jaggannath, Karnataka (IN); Chandrasekhar Bhaskaran Nair, Karnataka (IN); Pillarisetti Venkata Subbarao, Karnataka (IN)

(73) Assignee: BIGTEC PRIVATE LIMITED, Bangalore, Karnataka (IN)

(21) Appl. No.: 13/201,215

(22) PCT Filed: Jan. 27, 2010
(86) PCT No.: PCT/IN10/00048

§ 371 Date: Aug. 11, 2011

(30) Foreign Application Priority Data
Feb. 13, 2009 (IN) 00314/CHE/2009

(51) Int. Cl. C12Q 1/70; C07H 21/04

(52) U.S. Cl. 435/5; 536/24.32; 536/24.33

(57) ABSTRACT

The present disclosure provides a method for the detection and quantification of Hepatitis B Virus. It discloses oligonucleotide probes set forth in SEQ ID Nos. 1 and 2 for detection of Hepatitis B Virus along with respective primers [sense and antisense] set forth in SEQ ID Nos. 3, 4, 5 and 6. It also provides a PCR reaction mixture for detection of Hepatitis B Virus and a kit for detection of HBV comprising said mixture along with an instruction package.

### TECHNICAL FIELD

The present disclosure relates to method of determining presence and quantification of HBV (Hepatitis B virus) nucleic acids in samples.

### BACKGROUND AND PRIOR ART

HBV causes acute and chronic hepatitis (type B hepatitis), and in severe cases cirrhosis and hepatic carcinoma. Recent studies have shown that throughout the world number of people infected with hepatitis B virus (HBV) which is amounting to about 300 million.

PCR based assays for the direct detection of HBV nucleic acids in the blood/serum or plasma of an infected subject may provide an advantage in determining the exact viral load of an infected patient which will be useful for a physician to know the exact stage of infection. This may further help the physician to provide a proper therapy for the patient. Quantifying the exact viral load can also help in monitoring the progress of anti-viral therapy. The currently used methods for the diagnosis of HBV is based on ELISA (Enzyme Linked immune sorbent assay) which are based on the presence of serum markers such as, HbeAg, HbsAg, or anti-HBc IgM, anti-HBe, anti-HBs, or anti-HBc IgGs. Since ELISA based methods cannot give an insight into the exact viral load there is a need to look for a method which can give the quantitative measure of the viral load. There is a need for an effective method in view of the aforementioned problems associated with known methods of detection, so that the same can be utilized for detecting HBV which can give both qualitative and quantitative measure of the viral load.

### OBJECTIVES

First objective of the present disclosure is to provide a method to determine the presence of HBV nucleic acids in the samples.

Second objective of the present disclosure is to provide probes and primers for the detection of HBV.

Third objective of the present disclosure is to provide a PCR reaction mixture for the detection of HBV.

Fourth objective of the present disclosure is to provide a kit comprising probes and primers for the detection of HBV.

### BRIEF DESCRIPTION OF THE ACCOMPANYING DRAWINGS

FIG. 1 Real time plot of HBV positive samples using commercial kit

FIG. 2 Real time plot of HBV positive samples using SEQ ID No. 1

FIG. 3 Real time plot of HBV positive samples using SEQ ID No. 2

FIG. 4 Real time plot of HBV negative samples using SEQ ID No. 1

FIG. 5 Real time plot of HBV negative samples using SEQ ID No. 2

FIG. 6 HBV-Standard curve

### STATEMENT OF THE DISCLOSURE

Accordingly, the present disclosure is in relation to oligonucleotide probes of SEQ ID No. 1 and SEQ ID No. 2; Primers of SEQ ID Nos. 3, 4, 5 and 6; a PCR reaction mixture for detection of Hepatitis B Virus, said mixture comprising nucleic acid amplification reagents, dual labeled probes set forth in SEQ ID No. 1 and SEQ ID No.2, primers of SEQ ID Nos. 3, 4, 5, 6 and test sample; a method of detecting Hepatitis B Virus, said method comprising steps of: forming a reaction mixture comprising nucleic acid amplification reagents, oligonucleotide probe of SEQ ID No. 1 or SEQ ID No.2 with corresponding primers of SEQ ID Nos. 3 and 4 or SEQ ID Nos. 5 and 6 respectively, a test sample; and subjecting the reaction mixture to PCR to obtain copies of the target sequence followed by measuring the increase in fluorescence signal for detecting the Hepatitis B Virus; and a kit for detection of Hepatitis B Virus, said kit comprising dual labeled probes of SEQ ID No. 1 and SEQ ID No. 2, individually or in combination; corresponding pair of primers of SEQ ID Nos.3, 4, and 5, 6, individually or in combination and amplification reagents.

### DETAILED DESCRIPTION

The present disclosure is in relation to oligonucleotide probes of SEQ ID No. 1 and SEQ ID No. 2.

In one embodiment of the disclosure said probes are dual labeled probes.

In one embodiment of the disclosure said probes detect Hepatitis B Virus.

In one embodiment of the disclosure said probes are conjugated with detectable labels having fluorophore at 5′ end and a quencher in an internal region or at 3′ end.

In one embodiment of the disclosure the SEQ ID No. 1 is designed for surface gene of Hepatitis B virus and SEQ ID No. 2 is designed for X-gene region of Hepatitis B virus.

The present disclosure is in relation to primers of SEQ ID Nos. 3, 4, 5 and 6.

In one embodiment of the disclosure said primers of SEQ ID No.3 and SEQ ID No.5 are sense and SEQ ID No.4 and SEQ ID No.6 are anti sense primers respectively.

In one embodiment of the disclosure said primers of SEQ ID No.3 and SEQ ID No.4 are for dual labeled probe of SEQ ID No. 1 and primers of SEQ ID No.5 and SEQ ID No.6 are for dual labeled probe of SEQ ID No. 2.

The present disclosure is in relation to a PCR reaction mixture for detection of Hepatitis B Virus, said mixture comprising nucleic acid amplification reagents, dual labeled probes set forth in SEQ ID No. 1 and SEQ ID No.2, primers of SEQ ID Nos. 3, 4, 5, 6 and test sample.

In one embodiment of the disclosure said sample is selected from a group comprising blood, serum and plasma.

In one embodiment of the disclosure said PCR is real time PCR.

The present disclosure is in relation to a method of detecting Hepatitis B Virus, said method comprising steps of:
(a) forming a reaction mixture comprising nucleic acid amplification reagents, oligonucleotide probe of SEQ ID No. 1 or SEQ ID No.2 with corresponding primers of SEQ ID Nos. 3 and 4 or SEQ ID Nos. 5 and 6 respectively, a test sample; and
(b) subjecting the reaction mixture to PCR to obtain copies of the target sequence followed by measuring the increase in fluorescence signal for detecting the Hepatitis B Virus.

In one embodiment of the disclosure said probes are conjugated with detectable labels having fluorophore at 5′ end and a quencher in an internal region or at 3′ end.

In one embodiment of the disclosure said primers of SEQ ID No.3 and SEQ ID No.5 are sense and SEQ ID No.4 and SEQ ID No.6 are anti sense primers respectively.

In one embodiment of the disclosure said test sample is selected from a group comprising blood, serum and plasma.

In one embodiment of the disclosure said amplification reagents include magnesium chloride, Taq polymerase and buffer for amplification.

In one embodiment of the disclosure said detection is qualitative or quantitative in nature.

In one embodiment of the disclosure said fluorophore is selected from a group comprising fluorescein and fluorescein derivatives FAM, VIC, JOE, 5-(2′-aminoethyl)aminonaphthalene-1-sulphonic acid, coumarin and coumarin derivatives, lucifer yellow, texas red, tetramethylrhodamine, 6-Carboxy Fluorescein, tetrachloro-6-carboxyfluoroscein, 5-carboxyrhodamine and cyanine dyes.

In one embodiment of the disclosure said quencher is selected from a group comprising Tetra Methyl Rhodamine [TAMRA], 4′-(4-dimethylaminophenylazo) benzoic acid, 4-dimethylaminophenylazophenyl-4′-maleimide, tetramethylrhodamine, carboxytetramethylrhodamine and BHQ dyes.

In one embodiment of the disclosure said fluorophore is preferably 6-Carboxy Fluorescein at 5′ end and said quencher is preferably tetra methyl rhodamine at 3′ end or Black hole quencher 1 [BHQ1] in the internal region or at the 3′ end.

The present disclosure is in relation to a kit for detection of Hepatitis B Virus, said kit comprising dual labeled probes of SEQ ID No. 1 and SEQ ID No. 2, individually or in combination; corresponding pair of primers of SEQ ID Nos.3, 4, and 5, 6, individually or in combination and amplification reagents.

In one embodiment of the disclosure said amplification reagents include magnesium chloride, Taq polymerase and buffer for amplification.

### List of Biological Sequences of the Disclosure

SEQ ID No. 1 and the corresponding primers 3 and 4 have sequence identification numbers as shown in Table 1 below:

TABLE 1

| SEQUENCE | |
|---|---|
| ID NUMBER | NUCLEOTIDE SEQUENCE |
| SEQ ID | 5′-FAM-CCTCAGTCCGTTTCTCCTGGCTCAGTT- |
| No. 1 | TAMRA-3′ |
| | Or |
| | 5′-FAM-CCTCAGTCCGTT/iBHQ1T/ |
| | CTCCTGGCTCAGTT-Phos-3′ |
| SEQ ID | 5′-TGCACCTGTATTCCCATCCC-3′ |
| No. 3 | |
| SEQ ID | 5′-CCACATCATCCATATAACTGAAAGCC-3′ |
| No. 4 | |

SEQ ID No. 2 and the corresponding primers 5 and 6 have sequence identification numbers as shown in Table 2

TABLE 2

| SEQUENCE ID | |
|---|---|
| NUMBER | NUCLEOTIDE SEQUENCE |
| SEQ ID No. 2 | 5′-FAM-CCCCTTCTTCGTCTGCCGT-TAMRA-3′ |
| | Or |
| | 5′-FAM-CCCCTTCTTCG/iBHQ1T/CTGCCGT- |
| | Phos-3′ |
| SEQ ID No. 5 | 5′-CGTCGGCGCTGAATCC-3′ |
| SEQ ID No. 6 | 5′-GAAGCGAAGTGCACACGG-3′ |

The designed “Oligonucleotide” probes can be used for the detection of HBV nucleic acids in an infected sample by employing Real time PCR. The mode of detection is by measuring the increase in fluorescence during PCR.

HBV data base was thoroughly searched for identifying most conserved regions specific to HBV genome. The most promising regions with conserved regions were selected for designing the primer and probe sets. Conserved regions with in Surface and X genes were obtained and analyzed for designing PROBES and Primers.

According to the present invention SEQ ID No. 1 along with its respective sense and antisense primers of SEQ ID No. 3 and SEQ ID No. 4 are designed for the Surface gene of HBV genome. Similarly SEQ ID No. 2 along with its corresponding sense and antisense primers of SEQ ID No. 5 and SEQ ID No. 6 are designed for X gene of HBV genome.

According to the present invention said “Oligonucleotide” of SEQ ID No. 1 and SEQ ID No. 2 having detectable label with fluorophore at 5′ end and quencher in the internal region or at the 3′ end. The fluorophore is selected from a group comprising fluorescein and fluorescein derivatives FAM, VIC, JOE, 5-(2′-aminoethyl)aminonaphthalene-1-sulphonic acid, coumarin and coumarin derivatives, lucifer yellow, texas red, tetramethylrhodamine, 6-Carboxy Fluorescein, tetrachloro-6-carboxyfluoroscein, 5-carboxyrhodamine and cyanine dyes.

In still another embodiment of the present invention said quencher is selected from a group comprising Tetra Methyl Rhodamine, 4′-(4-dimethylaminophenylazo) benzoic acid, 4-dimethylaminophenylazophenyl-4′-maleimide, tetramethylrhodamine, carboxytetramethylrhodamine and BHQ dyes. The said fluorophore is preferably 6-Carboxy Fluorescein [FAM] and the quencher is Black hole quencher 1 [BHQ1] when present internally and Tetra Methyl Rhodamine [TAMRA] or Black hole quencher 1 [BHQ1] when present at the 3′ end.

The present invention is in relation to a method for detecting Hepatitis B Virus, where in the said PCR mixture comprising of nucleic acid amplification reagents, “Oligonucleotide” probes designated as SEQ ID No. 1 or SEQ ID No. 2, along with their corresponding primers of SEQ ID Nos. 3, 4, 5 and 6 and a test sample is subjected for amplification using real-time PCR to obtain copies of the target sequence. The amplification is measured in terms of increase in fluorescence signal.

The “Oligonucleotide” probe has a size ranging from 19-27 nucleotides. The designed probes have a fluorophore at the 5′ end and a quencher in the internal region or at the 3′ end.

The said fluorophore is preferably 6-Carboxy Fluorescein [FAM] and the quencher is Black hole quencher 1 [BHQ1] when present internally and Tetra Methyl Rhodamine [TAMRA] or Black hole quencher 1 [BHQ1] when present at the 3′ end. The current invention is used for the detection of hepatitis B virus present in blood/serum/plasma samples. The method used for detection is by monitoring the increase in fluorescence during the PCR.

According to the present invention the Oligonucleotide” probe refers to a short sequence of ribonucleic acid (RNA) or deoxyribonucleic acid (DNA). The “Oligonucleotide” probes can specifically hybridise to nucleic acids from all hepatitis B virus (HBV) genotypes. The “Oligonucleotide” probes according to the present invention is generally between about 19-27 nucleotides in length. The “Oligonucleotide” probes mentioned here specifically hybridise to the HBV nucleic acid sequence without exhibiting non-specific hybridisation to non-HBV nucleic acids. The “Oligonucleotide” sequence probes employed here follows the principles of Taqman chemistry. TaqMan probes also called Double-Dye oligonucleotide or dual labeled probes, are the most widely used type of probes. They were developed by Roche [Basel, Switzerland] and ABI [Foster City, USA] from an assay that originally used a radiolabeled probe and consist of a single-stranded probe sequence that is complementary to one of the strands of the amplicon. The fluorophore when excited passes its energy, via FRET (Fluorescence resonance energy transfer), to the quencher. During real time PCR the probe binds to the amplicon during each annealing step of the PCR. When the Taq polymerase extends from the primer bound to the amplicon it displaces the 5′ end of the probe, which is then degraded by the 5′-3′ exonuclease activity of the Taq polymerase. Cleavage continues until the remaining probe melts off the amplicon. This process releases the fluorophore and quencher into solution, specially separating them compared to when they were held together by the probe. This leads to an irreversible increase in fluorescence from the fluorophore.

The “Oligonucleotide” probes of SEQ ID Nos. 1 and 2 according to the present invention, therefore, is further provided in combination with their corresponding sense and antisense primers of SEQ ID Nos. 3, 4, 5 and 6 respectively, which can be used to specifically amplify and detect HBV nucleic acid sequences in a test sample by real time PCR.

The technology of the instant Application is further elaborated with the help of following examples. However, the examples should not be construed to limit the scope of the invention.

Efficacy and sensitivity of Oligonucleotide probes of SEQ ID No. 1 & SEQ ID No. 2 were analysed and compared with the commercial standard kit. Same concentrations of Real time PCR reagents, template and oligos were used in each case and also cycling conditions were kept constant for all the reactions. Based on the result obtained from the commercial standard kit the oligonucleotide probes of SEQ ID Nos. 1 and 2 were analyzed for their sensitivity and specificity.

### Example: 1

DNA was isolated from 10 HBV positive and 10 HBV negative serum samples using a commercial kit. Real time PCR reactions were carried out for all the samples using the Oligonucleotide probes designated as SEQ ID No. 1 and SEQ ID No. 2 along with their corresponding primers of SEQ ID Nos. 3, 4, 5 and 6 respectively. The sensitivity of these Oligonucleotide probes in picking up the infected samples was compared with a commercial standard kit. Same concentrations of Real time-PCR reagents, template and primers were used in each case and also cycling conditions were kept constant for all the reactions. The composition of the real time PCR mix and PCR conditions as given in Table 3 & 4.

TABLE 3
Real time-PCR with Takara Premix
Real time PCR Master Mix Composition

| | | |
|---|---|---|
| Premix | 5.0 | μl |
| Forward Primer | 0.2 | μl (2 picomoles) |
| Reverse Primer | 0.2 | μl(2 picomoles) |
| Probe | 0.2 | μl(2 picomoles) |
| Sample | 2.0 | μl |
| Total | 2.4 | μl |

TABLE 4
Real time-PCR cycle conditions
PCR Program

| | |
|---|---|
| Step 1 | 95° C. for 60 sec |
| Step 2 | 95° C. for 5 sec |
| Step 3 | 60° C. for 34 sec |

Step 2 and 3 will repeats 40 times

Results obtained showed that the Oligonucleotide probes designated as SEQ ID No. 1 and SEQ ID No. 2 picked up only the HBV positive samples and did not show any false amplification for the negative samples.

Oligonucleotide SEQ ID No. 1 picked up all the 10 positive samples within 40 cycles (positive sample cutoff) showing 100% specificity. Out of 10 detected positives, 9 samples were detected earlier when compared to commercial standard kit.

Similarly, oligonucleotide of SEQ ID No. 2 picked up all the 10 positive samples within 40 cycles (positive sample cutoff) showing 100% specificity. Out of 10 detected positives, 2 samples were detected earlier when compared to commercial standard kit.

Since SEQ ID No.1 picked many samples earlier than the commercial standard kit. Therefore, SEQ ID No. 1 is a better probe for screening HBV infections in terms of specificity and sensitivity. However both the oligonucleotide probes, SEQ ID No. 1 & SEQ ID No. 2 can be used for the detection of HBV infections.

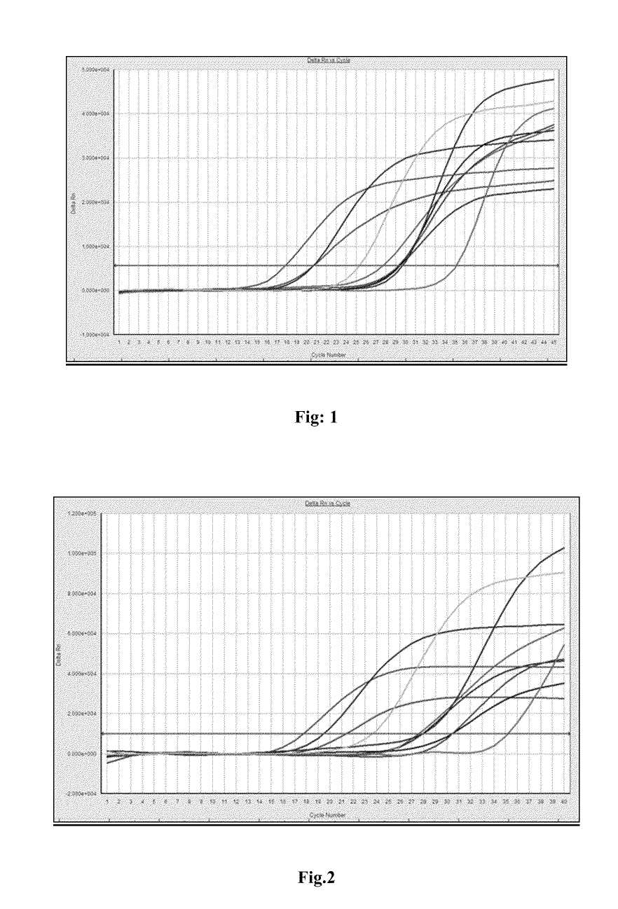

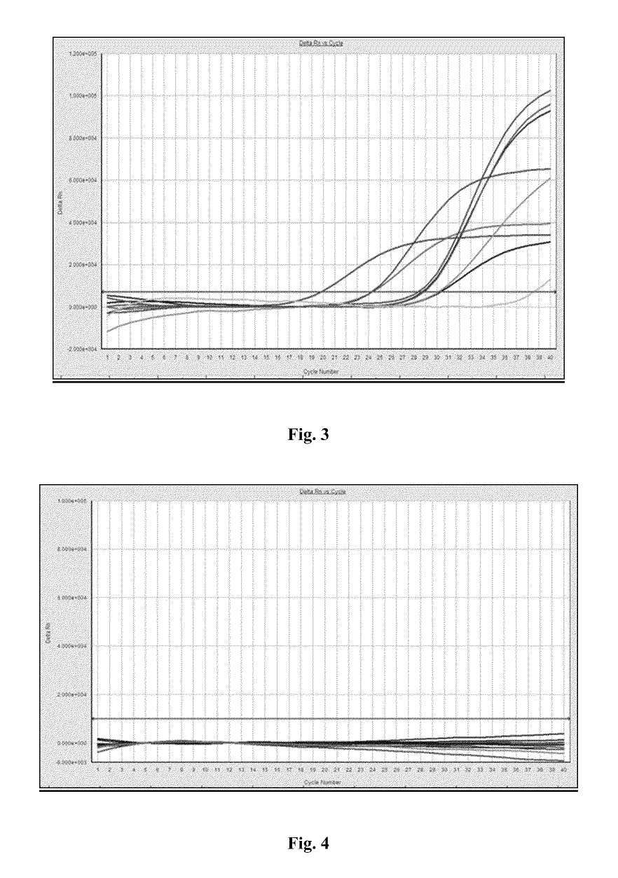

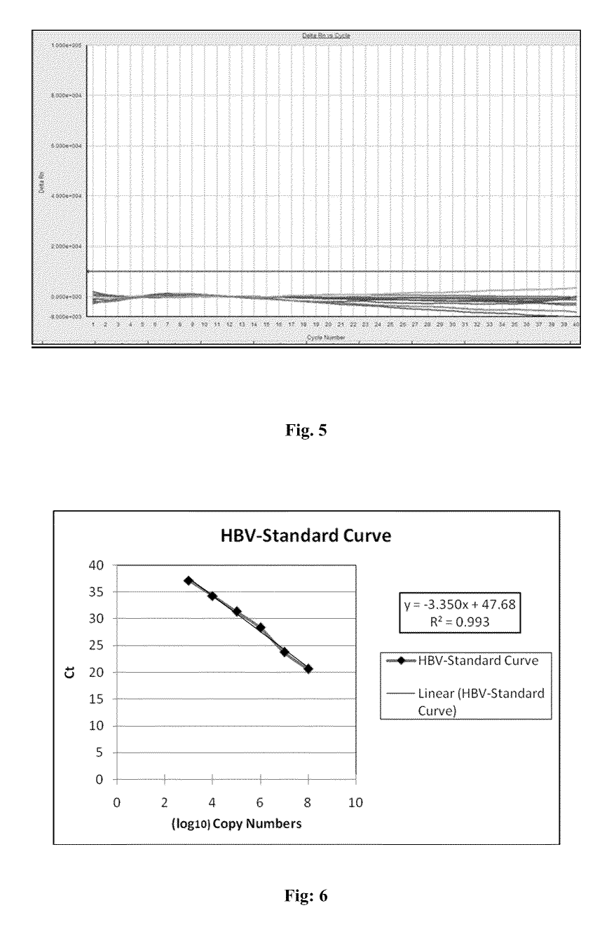

Table: 5 provides comparison of SEQ ID No.1 performance with Ct commercial kit. Similarly, Table: 6 provides comparison of SEQ ID No. 2 performance with Ct commercial kit. The real time PCR graphs of commercial kit, SEQ ID No.1 and SEQ ID No.2 are as given in FIGS. 1, 2, 3, 4 & 5.

TABLE 5
Comparison with commercial standard kit

| Sample ID | Ct commercial kit | Ct SEQ ID No. 1 |
|---|---|---|
| Positive 1 | 20.27 | 19.42 |
| Positive 2 | 17.48 | 17.22 |
| Positive 3 | 19.44 | 19.06 |
| Positive 4 | 32.03 | 26.04 |
| Positive 5 | 28.56 | 29.69 |
| Positive 6 | 27.37 | 23.73 |
| Positive 7 | 29.1 | 27.34 |
| Positive 8 | 30.12 | 27.99 |
| Positive 9 | 26.92 | 24.17 |
| Positive 10 | 37.46 | 35.11 |

TABLE 6
Comparison with commercial standard kit

| Sample ID | Ct commercial kit | Ct SEQ ID No. 2 |
|---|---|---|
| Positive 1 | 20.27 | 22.26 |
| Positive 2 | 17.48 | 20.18 |
| Positive 3 | 19.44 | 22 |
| Positive 4 | 32.03 | 30.46 |
| Positive 5 | 28.56 | 31.19 |
| Positive 6 | 27.37 | 29.65 |
| Positive 7 | 29.1 | 30.28 |
| Positive 8 | 30.12 | 28.64 |
| Positive 9 | 26.92 | 27.02 |
| Positive 10 | 37.46 | 39.20 |

### Example: 2

One can also quantify the viral load by generating a standard curve. For standard curve generation 25 μl of the HBV DNA was subjected to conventional PCR using a PCR mix containing dNTPS, Taq DNA polymerase, enzyme buffer, Mgcl₂ and primers specific for Surface and X gene regions. The conditions of the PCR are as follows:

### Step 1: 95° C. for 120 sec

### Step 2: 95° C. for 20 sec

### Step 3: 60° C. for 40 sec

Steps 2 & 3 were repeated for 40 cycles

After PCR the amplified sample was subjected to electrophoresis on a 3% agarose gel and stained with ethidium bromide. The amplicon band which is about 1.2 kbp length and which corresponds to the surface and the X gene regions of the HBV genome was then excised from the gel and purified using a Qiaquick gel extraction kit. The absorbance of the purified amplicon DNA (2 μl) was estimated at 260 nm using a nanodrop. Extinction coefficient of the DNA was calculated from individual base coefficient by summing up.

Nanomoles of amplicon were calculated using the following equation:

$$Nmoles \text{/} ml = \frac{1000 \times OD 260 ( 1 cm ) \times 1 ml ( vol )}{Extinction coefficient of amplicon}$$

Copy number was calculated using the formula:
Copy number/ml=(Moles/ml)×Avogadro number.

### Calculations:

### OD 260=0.522

Ext coefficient=33675.1
nmoles/ml=0.015501068

### Copies/ml=9.34×10¹²

From the copy number of the pure amplicon a standard curve was generated by running 10⁸ to 10³ dilutions of the amplicon using a real-time PCR. Composition of the real time PCR premix and PCR programme is as given in Table 3 & 4.

From the Ct obtained from the standard curve, FIG. 6, copy number can be calculated for unknown samples FIG. 6 & Table 7.

TABLE 7
Standard curve values with respect to Ct

| SI. No | Ct | Copies/ml |
|---|---|---|
| 1 | 20.6 | 1 × 10⁸ |
| 2 | 23.7 | 1 × 10⁷ |
| 3 | 28.3 | 1 × 10⁶ |
| 4 | 31.3 | 1 × 10⁵ |
| 5 | 34.2 | 1 × 10⁴ |
| 6 | 37.1 | 1 × 10³ |

### CONCLUSION

a) None of the negative samples showed false positive with the designed oligonucleotide probes designated as SEQ ID No. 1 & SEQ ID No. 2.
b) Oligonucleotide of SEQ ID No.1, which is designed for Surface gene of HBV, showed good specificity and sensitivity (100%). Out of 10 positive samples 9 were picked earlier than the commercial standard kit, Table 5.
c) Oligonucleotide SEQ ID No. 2, which is designed for X gene of HBV, also picked up all the 10 positives showing 100% specificity. Out of 10 positive samples 2 were picked earlier than the commercial standard kit, Table 6.
d) Based on the overall evaluation studies, SEQ ID No. 1 and SEQ ID No. 2 were considered to be the best for HBV detection vis-a-vis the commercial kits.
e) Finally the oligonucleotide probes designated as SEQ ID No. 1 & SEQ ID No. 2 can be used for quantifying viral load in an infected sample.

We claim:

1. Oligonucleotide probes as set forth in SEQ ID No. 1 or SEQ ID No.2.

2-22. (canceled)

23. The probes as claimed in claim 1, wherein said probes are dual labeled probes and are conjugated with detectable labels having fluorophore at 5′ end and a quencher in an internal region or at 3′ end.

24. The probes as claimed in claim 1, wherein said probes detect Hepatitis B Virus; and the SEQ ID No. 1 is designated for surface gene of Hepatitis B virus and SEQ ID No. 2 is designated for X-gene region of Hepatitis B virus.

25. Primers as set forth in SEQ ID Nos. 3, 4, 5 or 6.

26. The primers as claimed in claim 4, wherein said primers of SEQ ID No.3 and SEQ ID No.5 are sense and SEQ ID No.4 and SEQ ID No.6 are anti sense primers respectively.

27. The primers as claimed in claim 4, wherein said primers of SEQ ID No.3 and SEQ ID No.4 are for dual labeled probe of SEQ ID No. 1 and primers of SEQ ID No.5 and SEQ ID No.6 are for dual labeled probe of SEQ ID No. 2.

28. A PCR reaction mixture for detection of Hepatitis B Virus, said mixture comprising nucleic acid amplification reagents, dual labeled probes as set forth in SEQ ID No. 1 or SEQ ID No.2, corresponding primers as set forth in SEQ ID Nos. 3 and 4 or SEQ ID Nos. 5 and 6 respectively, and test sample.

29. The PCR reaction mixture as claimed in claim 7, wherein said sample is selected from a group comprising blood, serum and plasma; and said mixture is used in real time PCR.

30. A method of detecting Hepatitis B Virus, said method comprising steps of:
(a) forming a reaction mixture comprising nucleic acid amplification reagents, oligonucleotide probe as set forth in SEQ ID No. 1 or SEQ ID No.2 with corresponding primers as set forth in SEQ ID Nos. 3 and 4 or SEQ ID Nos. 5 and 6 respectively, a test sample; and
(b) subjecting the reaction mixture to PCR to obtain copies of the target sequence followed by measuring the increase in fluorescence signal for detecting the Hepatitis B Virus.

31. The method as claimed in claim 9, wherein said probes are conjugated with detectable labels having fluorophore at 5′ end and a quencher in an internal region or at 3′ end.

32. The method as claimed in claim 9, wherein said primers of SEQ ID No.3 and SEQ ID No.5 are sense and SEQ ID No.4 and SEQ ID No.6 are anti sense primers respectively.

33. The method as claimed in claim 9, wherein said test sample is selected from a group comprising blood, serum and plasma; and said amplification reagents include magnesium chloride, Taq polymerase and buffer for amplification.

34. The method as claimed in claim 9, wherein said detection is qualitative or quantitative in nature.

35. The method as claimed in claim 10, wherein said fluorophore is selected from a group comprising fluorescein and fluorescein derivatives FAM, VIC, JOE, 5-(2′-aminoethyl)aminonaphthalene-1-sulphonic acid, coumarin and coumarin derivatives, lucifer yellow, texas red, tetramethylrhodamine, 6-Carboxy Fluorescein, tetrachloro-6-carboxyfluoroscein, 5-carboxyrhodamine and cyanine dyes, preferably 6-Carboxy Fluorescein; and said quencher is selected from a group comprising Tetra Methyl Rhodamine [TAMRA], 4′-(4-dimethylaminophenylazo)benzoic acid, 4-dimethylaminophenylazophenyl-4′-maleimide, tetramethylrhodamine, carboxytetramethylrhodamine and BHQ dyes, preferably tetra methyl rhodamine at 3′ end or Black hole quencher 1 [BHQ1] in the internal region or at the 3′ end.

36. A kit for detection of Hepatitis B Virus, said kit comprising dual labeled probes as set forth in SEQ ID No. 1 and SEQ ID No. 2, individually or in combination; corresponding pair of primers as set forth in SEQ ID Nos.3, 4, and 5, 6, individually or in combination and amplification reagents.

37. The kit as claimed in claim 15, wherein said amplification reagents include magnesium chloride, Taq polymerase and buffer for amplification.
